(19) United States
(12) Patent Application Publication
(10) Pub. No.: US 2022/0240944 A1
(43) Pub. Date: Aug. 4, 2022

(54) ATHLETIC GARMENT WITH BLOOD FLOW OCCLUSION BANDS

(71) Applicants: Nicholas James Iconomou, Regentville (AU); Sonja Carissa Deacon, Kiama (AU)

(72) Inventors: Nicholas James Iconomou, Regentville (AU); Sonja Carissa Deacon, Kiama (AU)

(21) Appl. No.: 17/589,656

(22) Filed: Jan. 31, 2022

Related U.S. Application Data
(60) Provisional application No. 63/143,186, filed on Jan. 29, 2021.

(51) Int. Cl. A61B 17/132; A41D 13/00

(52) U.S. Cl. CPC A61B 17/1322; A41D 2300/33; A41D 2600/10; A41D 13/0015

(57) ABSTRACT

An athletic garment has a garment body with a limb portion extending therefrom. A lateral sleeve is formed on the limb portion, the lateral sleeve having a first sheet and a second sheet that are connected together to form a strap channel that extends at least partially around the limb portion, and includes an opening through the lateral sleeve that allows access to the strap channel. A blood flow occlusion band includes an elongate strap that is fastened with a fastener, the elongate strap being positioned within the strap channel of the lateral sleeve, such that the fastener is positioned in the opening of the lateral sleeve of the garment.

### CROSS-REFERENCE TO RELATED APPLICATIONS

This application for a utility patent claims the benefit of U.S. Provisional Application No. 63/143,186, filed Jan. 29, 2021.

### BACKGROUND OF THE INVENTION

### Field of the Invention

This invention relates generally to workout garments, and more particularly to a garment with integral blood flow occlusion bands.

### Description of Related Art

Blood flow occlusion bands are known in the art, in the form of inelastic bands that are positioned around a user's limb for occlusion of blood flow for purposes of increasing the effectiveness of a physical workout (e.g., weight lifting, cardio workouts, etc.).

Adducci, U.S. 2007/0287613, teaches clothing that incorporates inflatable bands for restricting blood flow. The device includes an inflatable band member that is controlled electronically via a pressure-inducing device and a pressure control device controlled by an electronic controller.

Osborn, U.S. 2020/0113578, similarly teaches clothing that includes a compression band sewn thereupon. The compression band include flexible air bladders and associated air assemblies to provide compression and blood flow moderation in a variety of applications including blood flow restriction training (BFR) and limb compression for injury prevention. The air assembly comprises a flexible manual pump, a check valve air intake, one-way valve and a pressure release valve. The user can properly position and fasten the air bladder around a limb using a special type of band, sleeve, or by wearing a piece of clothing containing strategically placed air bladders and integrated air assemblies. Each air assembly allows the user to easily and manually add air to the bladder by pressing on a designated area of the pump which causes the bladder to extend on the inward facing portion of the band, compression sleeve, or piece of clothing toward the limb until a desired pressure is achieved around the limb to support blood flow moderation.

There are also many very similar garments for military use, and utilize tourniquets for lifesaving treatment of injuries. This concept is quite old, with one example shown in Hobson, U.S. Pat. No. 2,702,551 (1951). Carabajal, U.S. 2019/0307465, teaches a wearable device adapted for military use that includes a tourniquet configured to occlude blood flow in an artery of a wearer. The tourniquet includes a base plate, two anchor points, and a tightening mechanism connected to the base plate. Similar tourniquet systems are also shown in Rose, U.S. Pat. No. 9,730,703, and Reynolds, U.S. Pat. No. 8,707,468.

The prior art teaches blood flow occlusion bands, and the prior art teaches sewing a blood flow occlusion band onto the sleeve of a garment. However, the prior art does not teach a blood flow occlusion band that is integrated into a lateral sleeve of the garment, which includes an opening for accessing a fastener such as a buckle. The present invention fulfills these needs and provides further advantages as described in the following summary.

### SUMMARY OF THE INVENTION

The present invention teaches certain benefits in construction and use which give rise to the objectives described below.

The present invention provides an athletic garment that includes a garment body with a limb portion extending therefrom, the limb portion including an upper portion adjacent the garment body, and a lower portion that extends to cover at least part of the limb of the user. A lateral sleeve is formed on the limb portion, the lateral sleeve having a first sheet and a second sheet that are connected together in first and second lateral areas that form, between the first and second sheets and the first and second lateral areas, a strap channel that extends around the limb portion, and includes an opening through the sleeve that allows access to the strap channel. A blood flow occlusion band includes an elongate strap that is fastened with a fastener, the elongate strap being positioned within the strap channel of the lateral sleeve, such that the fastener is positioned in the opening of the lateral sleeve of the garment.

A primary objective of the present invention is to provide an athletic garment having advantages not taught by the prior art.

Another objective is to provide an athletic garment that includes a blood flow occlusion band that is integrated into a lateral sleeve of the garment, which includes an opening for accessing a fastener.

A further objective is to provide an athletic garment that is attractive, easy to use, and also fully functional for blood flow occlusion training.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the present invention.

FIG. 1 is a front perspective view of a first embodiment of an athletic garment with blood flow occlusion bands, wherein the blood flow occlusion bands are integrated into legs of yoga pants.

FIG. 2 is a rear perspective view thereof.

FIG. 3 is a perspective view of a blood flow occlusion band apart from the athletic garment of FIG. 1.

FIG. 4 is a section view taken along lines 4-4 in FIG. 1.

FIG. 5 is a front elevation view of a second embodiment of the athletic garment, wherein the blood flow occlusion bands are integrated into sleeves of a shirt.

FIG. 6 is a front elevational view of a third embodiment of an athletic garment with blood flow occlusion bands.

FIG. 7 is an up-close view of a flap opening of the athletic garment of FIG. 6.

### DETAILED DESCRIPTION OF THE INVENTION

The above-described drawing figures illustrate the invention, an athletic garment with blood flow occlusion bands for intermittently engaging in venous occlusion training. Occlusion training may work biomechanically and neurophysiologically to alter pain perception, muscle activity, and blood circulation by restricting blood flow in the veins of a working muscle, which may in turn contribute to greater gains in muscle size and strength.

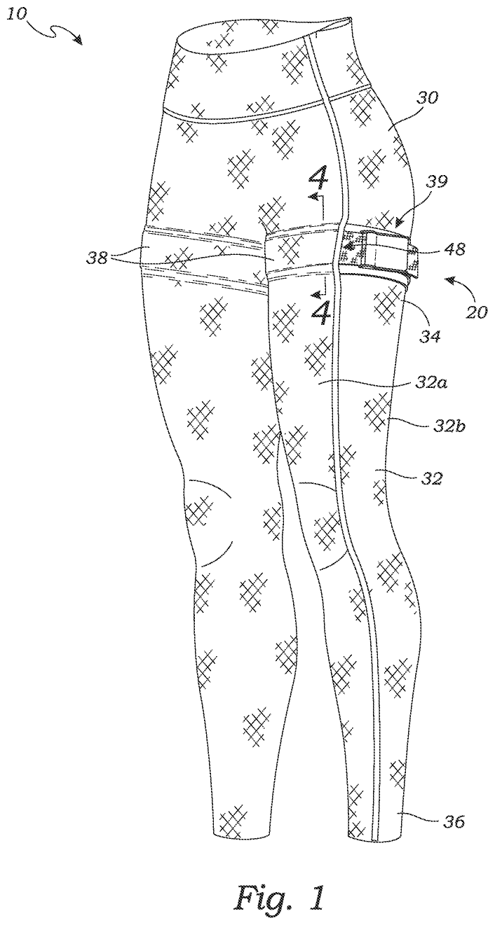

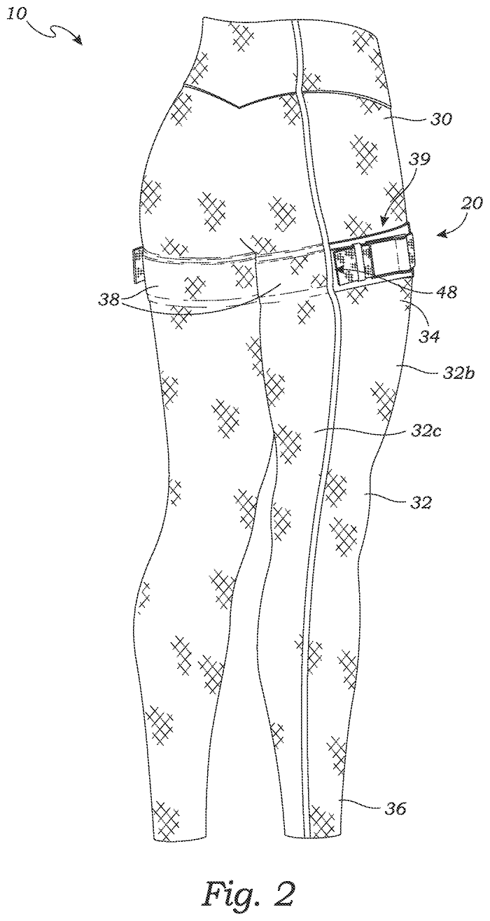

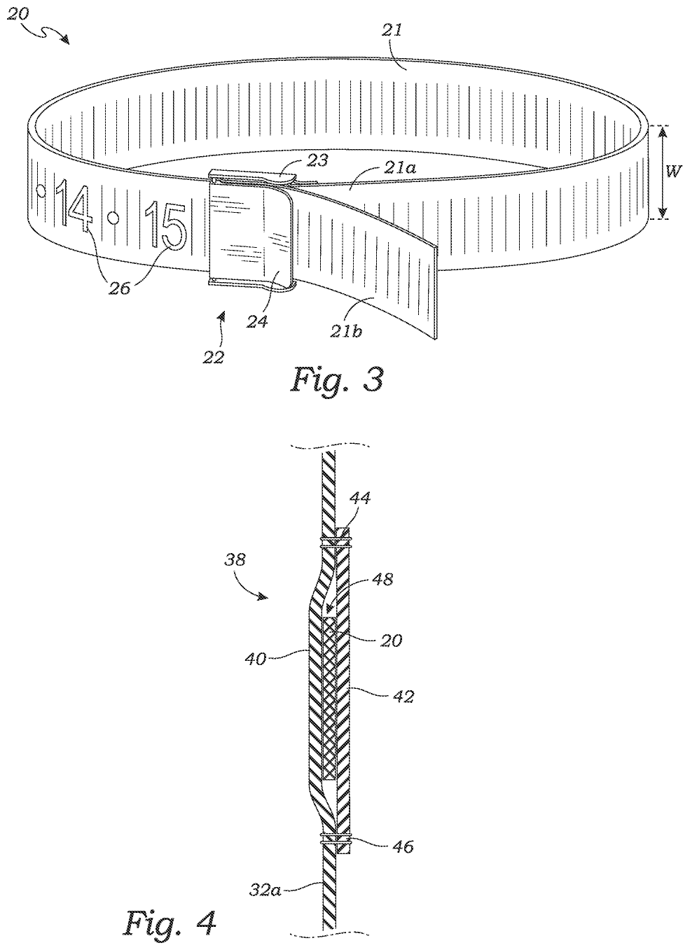

FIG. 1 is a front perspective view of a first embodiment of an athletic garment 10 with blood flow occlusion bands 20, wherein the blood flow occlusion bands 20 are integrated into legs of yoga pants. FIG. 2 is a rear perspective view thereof. As shown in FIGS. 1-2, the athletic garment 10 comprises a garment body 30 and limb portions 32 with blood flow occlusion bands 20 integrated into the limb portions 32 (i.e., sleeves, legs). The limb portions 32 include upper portions 34 adjacent the garment body 30, and lower portions 36 that extend at least part of the length of the leg of the user (or the arm, in alternative embodiments). The blood flow occlusion bands 20 may be integrated into the upper portions 34 of the limb portion 32 via a lateral sleeve 38 that is positioned adjacent to the garment body 30. The lateral sleeve 38 extends laterally, traverse to the limb of the user, at least partially around the limb portion 32, and includes an opening 39 through the lateral sleeve 38 that allows access to the blood flow occlusion band 20. This is shown in more detail in FIGS. 3-4, and discussed in greater detail below.

In this embodiment the garment body 30 is in the form of a pair of leggings (i.e., yoga pants). In alternative embodiments, the garment may be in the form of a shirt (as discussed below), shorts, jackets, or other similar form of garment that may be worn while engaging in athletic training. Further, the present invention may be provided in a set of athletic garments, such as a combination of shirt and leggings, each having at least one (typically a pair) of the blood flow occlusion bands 20.

In this embodiment, the garment includes at least a front panel 32a, a side panel 32b, and a rear panel 32c, although additional panels may be included, or entirely different configurations may be used, according to the requirements of the designed, without departing from the scope of the present invention. In this case, the front and side panels abut to form the opening 39 to the channel, and the side and rear panels also meet to form the other portion of the opening 39.

FIG. 3 is a perspective view of a blood flow occlusion band 20 apart from the athletic garment 10 of FIG. 1. As shown in FIG. 3, the blood flow occlusion band 20 includes an elongate strap 21 that is fastened with a fastener 22. In this embodiment, the fastener 22 is a buckle (e.g., a push down buckle), wherein the elongate strap 21 extends from a buckle end 21a to a free end 21b. The buckle end 21a may be attached to a base 23 of the buckle, and the free end 21b extends through a locking portion 24 that enables the elongate strap 21 to be secured in a loop that fits around the limb of the user, and the diameter of the loop may be adjusted by tightening or loosening the blood flow occlusion band 20. The buckle 22 may be larger than the strap channel 38 (shown in FIGS. 1, 2, and 4) so that it cannot enter the strap channel 38, and instead the buckle 22 is positioned in the opening 39 where it can be accessed by the user.

The width W of the elongate strap 21 is preferably about 2 inches, and the elongate strap 21 is preferably constructed of an elastic material, although in alternative embodiments, an inelastic material may be used. The elongate strap 21 may further include other features such as measurement indicia 26 printed onto the bands for measuring a perimeter length of the loop formed by the blood flow occlusion band 20, to track how tight the band is, and how much occlusion is being applied. For purposes of this application, the term “printed” is defined to include any form of printing, marking, embossing, stitching, or otherwise forming the indicia on the surface so that they are visible to the user.

FIG. 4 is a section view taken along lines 4-4 in FIG. 1, which illustrates the lateral sleeve 38 shown in FIGS. 1-2. As shown in FIG. 4, the lateral sleeve 38 includes a first sheet 40 and a second sheet 42 that are connected together in first and second lateral areas 44 and 46 (e.g., lateral stitches) that form, between the first and second sheets and the first and second lateral stitches, a strap channel 48, through which the elongate strap 21 of the blood flow occlusion band 20 is threaded. As shown in FIGS. 1-2, the buckle 22 remains outside of the strap channel 48, and is positioned in the opening 39, where it can be accessed by the user. The opening 39 shown in FIGS. 1-2 allows the elongate strap 21 to be inserted into the strap channel 48. The blood flow occlusion band 20 may be removed while cleaning the garment.

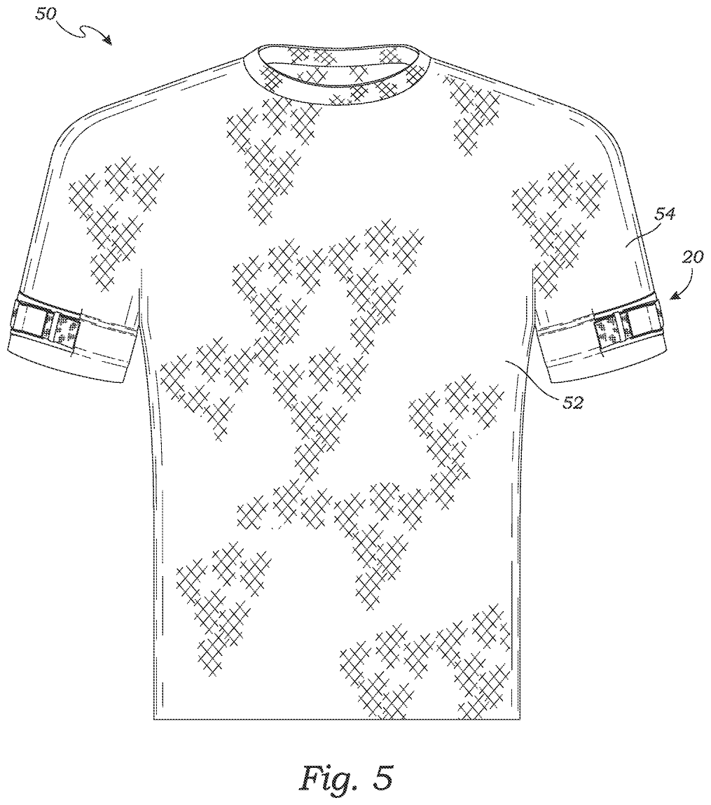

FIG. 5 is a front elevation view of a second embodiment of the athletic garment 50, wherein the blood flow occlusion bands 20 are integrated into sleeves of a shirt. As shown in FIG. 5, the shirt includes a main body 52 and a pair of sleeves 54 which extend outwardly, and the blood flow occlusion bands 20 are positioned in the sleeves, as shown. As illustrated by FIG. 5, any form of athletic garment 10, which includes sleeves or legs, may be adapted for this invention, and any alternative or equivalent garments should be considered within the scope of the present invention.

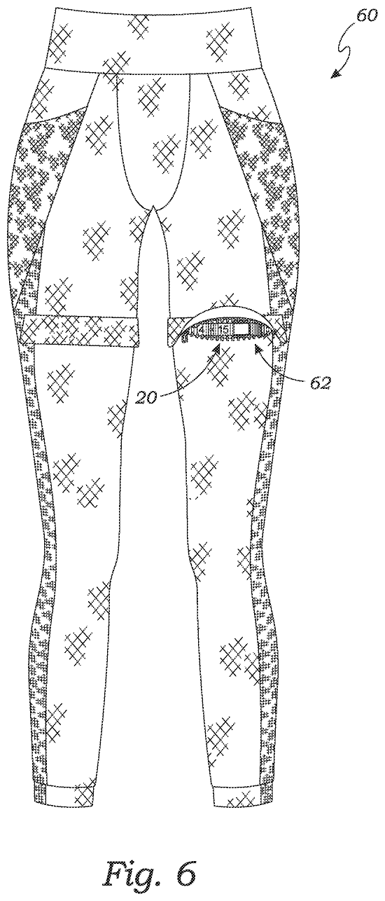

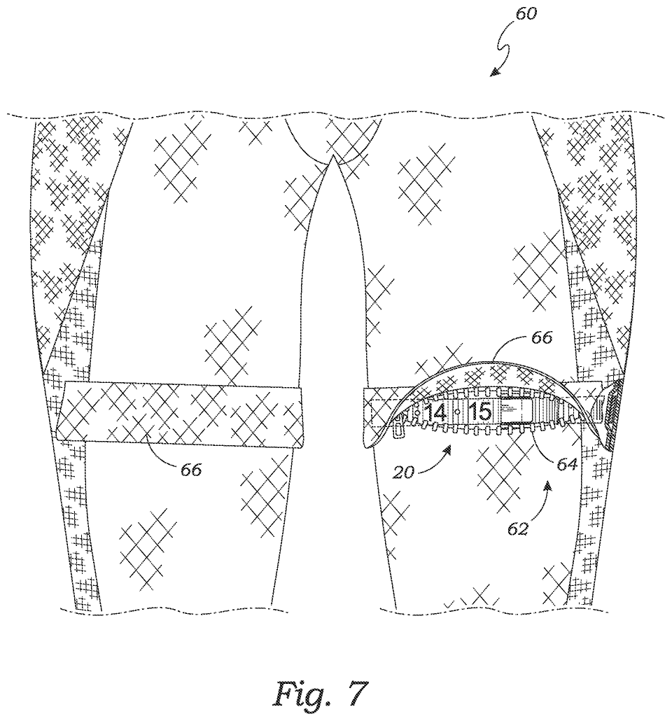

FIG. 6 is a front elevational view of a third embodiment of an athletic garment 60 having concealed blood flow occlusion bands 20. FIG. 7 is an up-close view of a concealed opening 62 of the athletic garment of FIG. 6. As shown in FIGS. 6-7, in this alternative embodiment, the blood flow occlusion bands 20 may be incorporated into a garment that includes a closure mechanism 64 (in this case, a zipper, although any suitable fastener known in the art may be used). Furthermore, the closure mechanism 64 may be concealed with a covering flap 66 that extends across the opening and the closure mechanism, to conceal them from external visibility while not in use.

As used in this application, the words “a,” “an,” and “one” are defined to include one or more of the referenced item unless specifically stated otherwise. Also, the terms “have,” “include,” “contain,” and similar terms are defined to mean “comprising” unless specifically stated otherwise. Furthermore, the terminology used in the specification provided above is hereby defined to include similar and/or equivalent terms, and/or alternative embodiments that would be considered obvious to one skilled in the art given the teachings of the present patent application.

What is claimed is:

1. An athletic garment for engaging in venous occlusion training in a limb of a user, the athletic garment comprising:
a garment body with a limb portion extending therefrom, the limb portion including an upper portion adjacent the garment body, and a lower portion that extends to cover at least part of the limb of the user;
a lateral sleeve that is formed on the upper portion of the limb portion, the lateral sleeve having a first sheet and a second sheet that are connected together in first and second lateral stitches that form, between the first and second sheets and the first and second lateral stitches, a strap channel that extends partially around the limb portion, and includes an opening through the sleeve that allows access to the strap channel; and
a blood flow occlusion band that includes an elongate strap that is fastened with a fastener, the elongate strap being positioned within the strap channel of the lateral sleeve, such that the fastener is positioned in the opening of the lateral sleeve. The athletic garment of claim 1, wherein the fastener is a buckle.

2. The athletic garment of claim 1, wherein the fastener is a push down buckle.

3. The athletic garment of claim 1, wherein the elongate strap extends from a buckle end to a free end; and wherein the buckle end is attached to a base of a buckle, and the free end extends through a locking portion that enables the elongate strap to be secured in a loop that fits around the limb of the user, and the diameter of the loop may be adjusted by tightening or loosening the blood flow occlusion band.

4. The athletic garment of claim 1, wherein the buckle is larger than the strap channel so that it cannot enter the strap channel, and wherein the buckle is positioned in the opening where it can be accessed by the user.

5. The athletic garment of claim 1, wherein the elongate strap is about 2 inches wide.

6. The athletic garment of claim 1, wherein the elongate strap is constructed of an elastic material.

7. The athletic garment of claim 1, wherein the elongate strap includes measurement indicia printed onto the bands for measuring a perimeter length of the loop formed by the blood flow occlusion band, to track how tight the band is, and how much occlusion is being applied.

8. An athletic garment for engaging in venous occlusion training in a limb of a user, the athletic garment comprising:
a garment body with a limb portion extending therefrom, the limb portion including an upper portion adjacent the garment body, and a lower portion that extends to cover at least part of the limb of the user;
a lateral sleeve that is formed on the limb portion, the lateral sleeve having a first sheet and a second sheet that are connected together to form a strap channel that extends at least partially around the limb portion, and includes an opening through the lateral sleeve that allows access to the strap channel; and
a blood flow occlusion band that includes an elongate strap that is fastened with a fastener, the elongate strap being positioned within the strap channel of the lateral sleeve, such that the fastener is positioned in the opening of the lateral sleeve. The athletic garment of claim 9, wherein the fastener is a buckle.

9. The athletic garment of claim 9, wherein the fastener is a push down buckle.

10. The athletic garment of claim 9, wherein the elongate strap extends from a buckle end to a free end; and wherein the buckle end is attached to a base of a buckle, and the free end extends through a locking portion that enables the elongate strap to be secured in a loop that fits around the limb of the user, and the diameter of the loop may be adjusted by tightening or loosening the blood flow occlusion band.

11. The athletic garment of claim 9, wherein the buckle is larger than the strap channel so that it cannot enter the strap channel, and wherein the buckle is positioned in the opening where it can be accessed by the user.

12. The athletic garment of claim 9, wherein the elongate strap is about 2 inches wide.

13. The athletic garment of claim 9, wherein the elongate strap is constructed of an elastic material.

14. The athletic garment of claim 9, wherein the elongate strap includes measurement indicia printed onto the bands for measuring a perimeter length of the loop formed by the blood flow occlusion band, to track how tight the band is, and how much occlusion is being applied.
